(19) United States
(12) Patent Application Publication
(10) Pub. No.: US 2026/0154093 A1
(43) Pub. Date: Jun. 4, 2026

(54) COGNITIVE LOAD EXPERIENCE

(71) Applicant: American Express Travel Related Services Company, Inc., New York, NY (US)

(72) Inventors: Sibish Basheer, Phoenix, AZ (US); André Kerr, Vernon, CT (US)

(21) Appl. No.: 18/965,160

(22) Filed: Dec. 2, 2024

(51) Int. Cl. G06F 9/451; A61B 5/16

(52) U.S. Cl. CPC G06F 9/451; A61B 5/165

(57) ABSTRACT

Disclosed are various embodiments for using cognitive load to modify a user experience. A computing device can monitor a plurality of user interactions associated with a user and identify an intended task associated with one or more user interactions of the plurality of user interactions. Next, the computing device can calculate an extrinsic load score based at least in part on the plurality of user interactions. Next, the computing device can conduct an analysis of the extrinsic load score based at least in part on the intended task and modify a user experience based at least in part on the analysis.

### BACKGROUND

With the growth of the digital world, more daily experiences are occurring in an online space. The human-computer interaction for a digital experience varies widely across individuals. For example, when a user has a digital interaction with an organization, much of the experience can vary based on the user's knowledge of computers, the organization, the platform, and the interaction itself.

Additionally, according to Hick's Law, the time it takes to make decisions increases with the number and complexity of choices. In digital experiences, a user could be subjected to both extraneous cognitive load and intrinsic cognitive load as they interact with computers to complete a task.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a drawing of a network environment according to various embodiments of the present disclosure.

FIG. 2 is a flowchart illustrating one example of functionality implemented as portions of an application executed in a computing environment in the network environment of FIG. 1 according to various embodiments of the present disclosure.

FIGS. 3-6 are flowcharts illustrating various examples of functionality implemented as portions of the application from FIG. 2 according to various embodiments of the present disclosure.

### DETAILED DESCRIPTION

Disclosed are various approaches for calculating the cognitive load of different users while completing a human-computer interaction. Load is imposed on the cognitive system of user when the user is performing a particular task. Various factors can impact this cognitive load, such as the user being introduced to new information, complexity of the digital experience, etc. Cognitive load can be categorized in a few different measures. For example, cognitive load can include extrinsic cognitive load and intrinsic cognitive load. Extrinsic cognitive load can be defined as the generic load imposed on the cognitive system by performing a particular task. This form of cognitive load is related to the task or experience itself. Extrinsic cognitive load can include unnecessary distractions in a digital experience which take attention away from the main task. For example, the relevancy of search results for a particular search can impact cognitive load, as well as factors such as the number of options presented to a user in order for them to make a decision.

In contrast, intrinsic cognitive load can be defined as the load imposed on the particular user's cognitive system by performing a particular task. Unlike extrinsic cognitive load, which is based on the experience itself, the intrinsic cognitive load is based on the user. For example, factors such as prior knowledge or experience with a particular task can reduce intrinsic cognitive load. Additionally, factors such as digital savviness of the user and personal preferences can also impact the intrinsic cognitive load.

Measuring extrinsic and intrinsic cognitive load for a particular user and experience can be a difficult undertaking. An interested party can attempt to measure cognitive load by conducting a post-experience interview. For example, a survey or questionnaire can be presented at the end of a digital interaction, asking the individual for feedback on the interaction. However, a questionnaire is unable to accurately measure cognitive load since it is extremely difficult or impossible to capture every detail of an interaction through a survey. Due to limitations on quantity/quality of questions as well as difficulty for the individual to accurately recall and relay every detail of the interaction, this method is not suited for measuring cognitive load.

In another example, measuring extrinsic cognitive load and intrinsic cognitive load can be attempted through use of Hick's Law by measuring the amount of time it takes a user to complete a task. However, this measurement cannot serve as a proxy for cognitive load since time is merely correlated with cognitive load. Indeed, this method fails to account for a variety of important factors which impact cognitive load and further fails to distinguish between extrinsic and intrinsic cognitive load.

Accordingly, the present disclosure relates to various approaches for calculating the extrinsic and intrinsic cognitive load of different users while completing a human-computer interaction. In addition, this disclosure provides for methods of using the calculated load to modify and personalize a user-experience. By using real-time measurement of a variety of details of a user-experience and combining this real-time data with broader historical data, the present method can use machine-learning to both calculate cognitive load and provide a path-forward for modifying a user-experience.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

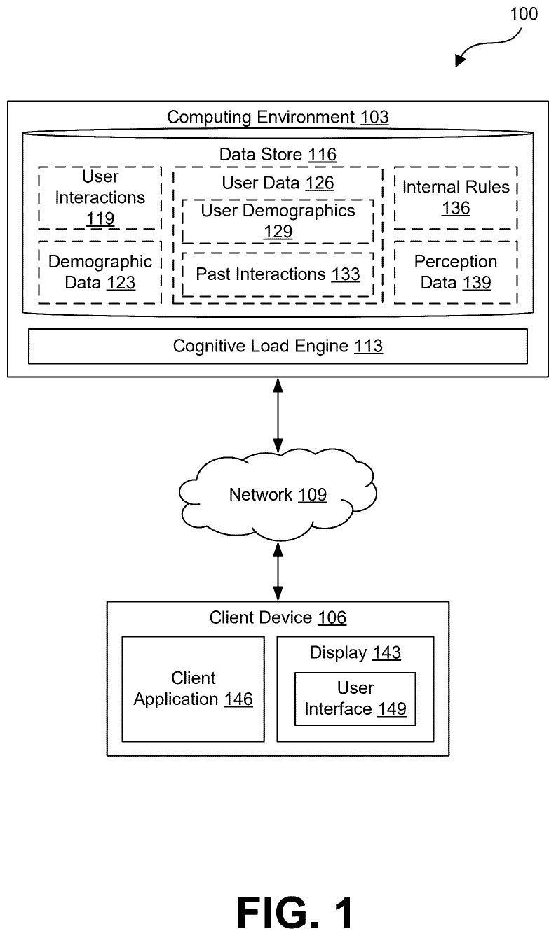

With reference to FIG. 1, shown is a network environment 100 according to various embodiments. The network environment 100 can include a computing environment 103 and a client device 106, which can be in data communication with each other via a network 109.

The network 109 can include wide area networks (WANs), local area networks (LANs), personal area networks (PANs), or a combination thereof. These networks can include wired or wireless components or a combination thereof. Wired networks can include Ethernet networks, cable networks, fiber optic networks, and telephone networks such as dial-up, digital subscriber line (DSL), and integrated services digital network (ISDN) networks. Wireless networks can include cellular networks, satellite networks, Institute of Electrical and Electronic Engineers (IEEE) 802.11 wireless networks (i.e., WI-FI®), BLUETOOTH® networks, microwave transmission networks, as well as other networks relying on radio broadcasts. The network 109 can also include a combination of two or more networks 109. Examples of networks 109 can include the Internet, intranets, extranets, virtual private networks (VPNs), and similar networks.

The computing environment 103 can include one or more computing devices that include a processor, a memory, and/or a network interface. For example, the computing devices can be configured to perform computations on behalf of other computing devices or applications. As another example, such computing devices can host and/or provide content to other computing devices in response to requests for content.

Moreover, the computing environment 103 can employ a plurality of computing devices that can be arranged in one or more server banks or computer banks or other arrangements. Such computing devices can be located in a single installation or can be distributed among many different geographical locations. For example, the computing environment 103 can include a plurality of computing devices that together can include a hosted computing resource, a grid computing resource or any other distributed computing arrangement. In some cases, the computing environment 103 can correspond to an elastic computing resource where the allotted capacity of processing, network, storage, or other computing-related resources can vary over time.

Various applications or other functionality can be executed in the computing environment 103. The components executed on the computing environment 103 include a cognitive load engine 113, and other applications, services, processes, systems, engines, or functionality not discussed in detail herein.

The cognitive load engine 113 can be executed to determine various cognitive load scores experienced by a user while interacting with an organization online. The cognitive load engine 113 can collect real-time data about a user experience and combine this data with previously-acquired data about the user. Together, this data can serve as the basis for cognitive load determinations. In some examples, the cognitive load engine 113 can use machine learning techniques to evaluate the data and weight various factors in order to calculate cognitive load scores. In addition, the cognitive load engine 113 can use the scores to suggest changes or make modifications to the user experience.

Also, various data is stored in a data store 116 that is accessible to the computing environment 103. The data store 116 can be representative of a plurality of data stores 116, which can include relational databases or non-relational databases such as object-oriented databases, hierarchical databases, hash tables or similar key-value data stores, as well as other data storage applications or data structures. Moreover, combinations of these databases, data storage applications, and/or data structures may be used together to provide a single, logical, data store. The data stored in the data store 116 is associated with the operation of the various applications or functional entities described below. This data can include user interactions 119, demographic data 123, user data 126 including user demographics 129 and past interactions 133, internal rules 136, perception data 139, and potentially other data.

The user interactions 119 can represent one or more real-time interactions of a user with a particular platform. In some examples, the user interactions 119 include various interactions with a user interface during a user's digital experience. User interactions 119 can include requests, searches, clicks, keystrokes, touch occurrences, mouse movements, eye movements, fixation on content, repeated interactions, number of channels accessed, time per journey, etc. For example, where a user attempts to complete a journey first through a mobile application and then through a web application, user interactions 119 would reflect that multiple channels were used before successful completion of the journey. In another example, where a user spends several seconds (e.g., 10 or more, 15 or more, 20 or more, etc.) on one page, the user interactions 119 would reflect a higher fixation on the content than if the user had spent only a few seconds (e.g., less than 10, less than 5, etc.) on the page.

The demographic data 123 can represent external data or knowledge about various groups of people and their experiences. Demographic data 123 used by the cognitive load engine 113 can include statistics for computer knowledge and skills across various groups, as well as familiarity with particular services or products. Demographic data 123 can include statistics about age, language, culture, race, gender, nationality, location, household, income, or various other categories.

The user data 126 can represent user-specific data including name, age, gender, account information (e.g., account number, account type, account history, etc.), and other information. In some examples, the user data 126 can include user demographics 129. The user demographics 129 can include information about which demographic groups the user falls within. For example, the user demographics 129 can include the age, gender, race, nationality, native language, location, income, household status, etc. of the user. The user data can include past interactions 133. Similar to user interactions 119, the past interactions 133 can include requests, searches, clicks, keystrokes, touch occurrences, mouse movements, eye movements, fixation on content, repeated interactions, number of channels accessed, time per journey, etc. However, rather than real-time interactions, the past interactions 133 are historical events for previous journeys that the user has completed.

Internal rules 136 can represent a set of rules, instructions, or directions which can be used in the analysis of various data. In some examples, the internal rules 136 are determined based at least in part on the structure and intent of the host organization. The internal rules 136 can be specific to an application, a task which the user is trying to complete, a particular subset of users, or other qualifier set by the host organization. The internal rules 136 can include “if-then” statements for the cognitive load engine 113 to use when modifying a user experience, as well as threshold values for the various scores which can also be used by the cognitive load engine 113 when making decisions.

Perception data 139 can represent data about a user's perception of the host organization. For example, perception data 139 can include prior surveys, feedback, social media reviews, or other forms of the user's sentiment on public forums. In addition, perception data 139 can include a user's history of engagement with particular services or products offered by the organization, whether they have ever referred other users to the organization, repeat interactions, etc. In some examples, perception data 139 can also include interactions with partner organizations, advertisements which have reached the user, etc. The perception data 139 can also include a variety of external factors which may influence a user's perception of an organization such as global and political events, sponsorships, charity affiliations, wars, natural catastrophes, or other events.

The client device 106 is representative of a plurality of client devices that can be coupled to the network 109. The client device 106 can include a processor-based system such as a computer system. Such a computer system can be embodied in the form of a personal computer (e.g., a desktop computer, a laptop computer, or similar device), a mobile computing device (e.g., personal digital assistants, cellular telephones, smartphones, web pads, tablet computer systems, music players, portable game consoles, electronic book readers, and similar devices), media playback devices (e.g., media streaming devices, BluRay® players, digital video disc (DVD) players, set-top boxes, and similar devices), a videogame console, or other devices with like capability. The client device 106 can include one or more displays 143 such as liquid crystal displays (LCDs), gas plasma-based flat panel displays, organic light emitting diode (OLED) displays, electrophoretic ink (“E-ink”) displays, projectors, or other types of display devices. In some instances, the display 143 can be a component of the client device 106 or can be connected to the client device 106 through a wired or wireless connection.

The client device 106 can be configured to execute various applications such as a client application 146 or other applications. The client application 146 can be executed in a client device 106 to access network content served up by the computing environment 103 or other servers, thereby rendering a user interface 149 on the display 143. To this end, the client application 146 can include a browser, a dedicated application, or other executable, and the user interface 149 can include a network page, an application screen, or other user mechanism for obtaining user input. The client device 106 can be configured to execute applications beyond the client application 146 such as email applications, social networking applications, word processors, spreadsheets, or other applications.

Next, a general description of the operation of the various components of the network environment 100 is provided. To begin, a user can participate in a digital experience, or human-computer interaction, via a client device 106. For example, a user can begin a journey with a particular organization through a mobile app, a web portal, or other digital avenue. The journey can be a search, a modification of an account, or other experience which includes multiple steps. A cognitive load engine 113, hosted by the organization, can monitor the user's journey in real time, and collect a variety of data about the user's experience, such as the user interactions 119. Once this data has been collected, the cognitive load engine 113 can reference user data 126, such as the user demographics 129 and past interactions 133 with the organization. Using this combination of data, the cognitive load engine 113 can calculate one or more scores representing the extrinsic and intrinsic cognitive load on the user during their digital journey. In some examples, the cognitive load engine 113 can also calculate a score for the user's perception of the organization. Next, the cognitive load engine 113 can use the scores and a set of internal rules 136 determined by the organization to customize the user experience going forward. In some examples, this process occurs in real time as the user is completing their digital interaction, and the cognitive load engine 113 can modify the user experience as it is happening.

In one example, a user searches for how to extend their credit limit for a credit card using the mobile app for the credit institution. The cognitive load engine 113 can monitor the search and the user's experience, collecting data such as content of the search, fixation on the content of the results, number of results with which the user interacted, number of clicks/touches, total time spent on the search, and eye movements of the user on the screen. Using this data, the cognitive load engine 113 can calculate an extrinsic load score and conduct an analysis to formulate reasons for the score. In some examples, the cognitive load engine 113 can calculate an extrinsic load score using a weighted sum of the number of user interactions 119 to produce a high number and note the highest weighted user interactions 119 as the reasons for the score. For example, the cognitive load engine 119 can assign high weights to factors such as fixation, low number of results with which the user interacted, and a high number of eye movements. These factors would be listed as reasons for a high extrinsic load score to indicate that the extrinsic load on the user was substantial.

Similarly, the cognitive load engine 113 can calculate an intrinsic load score using user data 126. For example, the cognitive load engine 113 can obtain user data 126 from a database and determine information such as the user's past interactions with the app, past interactions with similar apps, prior knowledge of the search engine within the app, as well as demographics 129 such as the user's birth generation, digital savviness, country, language, culture, location, etc. In some examples, the cognitive load engine 113 can use a weighted sum of various factors from the user data 126 to calculate the intrinsic cognitive load experienced by the user and formulate an explanation of the score. For example, the cognitive load engine 113 can determine that the user has a high number of mobile interactions, is new to the credit company, and originates from a country with a different language than is currently being used in the app. These factors can be weighted heavily to influence the outcome of the intrinsic load score and can be provided as reasons for the score.

Once the cognitive load engine 113 has determined the extrinsic load score and the intrinsic load score, the cognitive load engine 113 can use internal rules 136 to customize the user experience. In some examples, the cognitive load engine 113 determines that the extrinsic and intrinsic load scores are both high for the particular user and their experience. Using the internal rules 136, the cognitive load engine 113 can determine that the user experience must be modified to reduce the extrinsic and intrinsic cognitive load. For example, if a user has a high extrinsic load score when conducting their search for how to extend their credit limit, the cognitive load engine 113 can cause the user to be directed to a chat with an agent of the credit institution, bypassing a routine step of providing more search results. Similarly, in another example, if a user has a high intrinsic load score while conducting this search, the cognitive load engine 113 can cause the user to be directed to a chat with a human agent of the credit institution rather than an artificial intelligence chat bot.

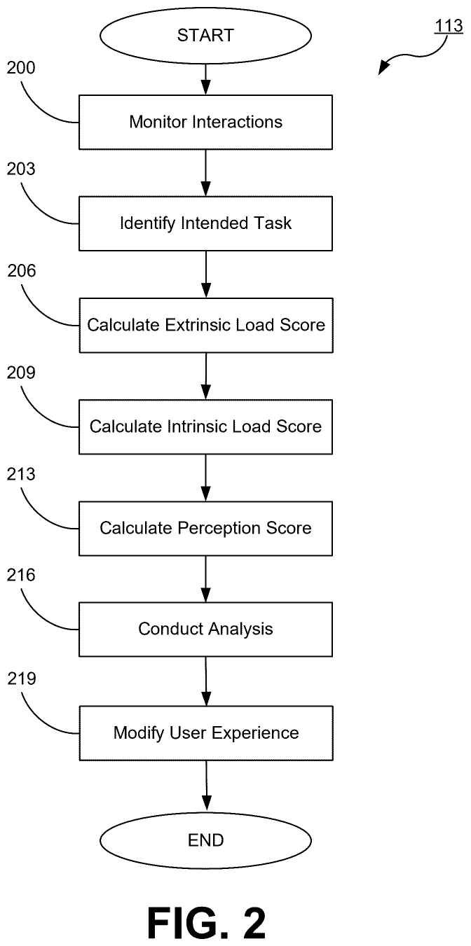

Referring next to FIG. 2, shown is a flowchart that provides one example of the operation of a portion of the cognitive load engine 113. The flowchart of FIG. 2 provides merely an example of the many different types of functional arrangements that can be employed to implement the operation of the depicted portion of the cognitive load engine 113. As an alternative, the flowchart of FIG. 2 can be viewed as depicting an example of elements of a method implemented within the network environment 100.

Beginning with block 200, the cognitive load engine 113 can be executed to monitor one or more user interactions 119. The cognitive load engine 113 can be in data communication with a client application 146 on a client device 106 over a network 109. The cognitive load engine 113 can receive real-time data about user interactions 119 with the client application 146. In some examples, the cognitive load engine 113 requests the user interactions 119 from the client application 146. In some examples, the client application 146 sends the user interactions 119 when a user begins a digital experience through the client application 146.

Next, at block 203, the cognitive load engine 113 can be executed to identify an intended task. Based at least in part on the user interactions 119 being monitored at block 200, the cognitive load engine 113 can identify an intended task of the user. In some examples, the cognitive load engine 113 can consult a list of tasks which are available through the client application 146 and identify an intended task based at least in part on the list of tasks. For example, if the user has completed a search and interacted with one or more results, the cognitive load engine 113 can refer to the list of tasks and identify at least one task which involves key words used in both the search and the selected results. The cognitive load engine 113 can determine that the user intends to complete the identified task.

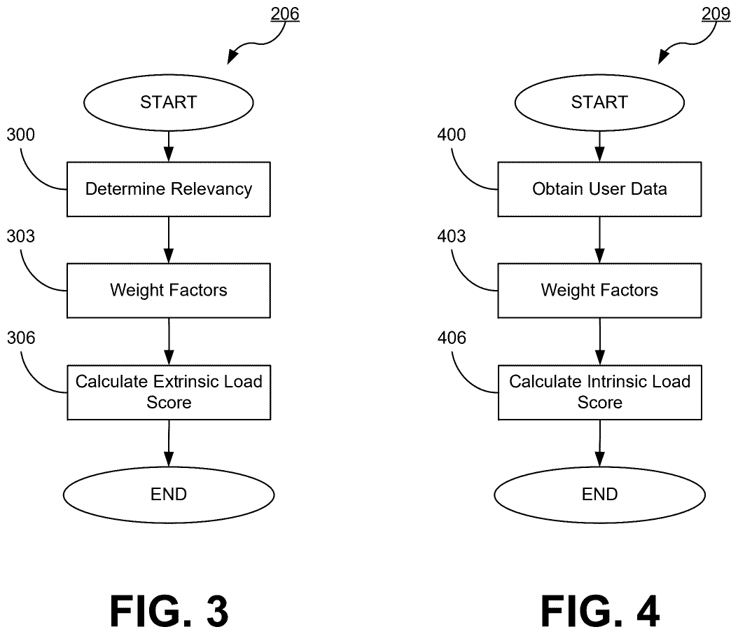

At block 206, the cognitive load engine 113 can be executed to calculate an extrinsic load score. The cognitive load engine 113 can calculate an extrinsic load score as a measure of the extrinsic cognitive load experienced by the user during the user interactions 119 from block 200. In some examples, the cognitive load engine 113 can calculate an extrinsic load score based at least in part on the user interactions 119 from block 200. The cognitive load engine 113 can calculate the extrinsic load score based at least in part on the task identified at block 203. In some examples, the cognitive load engine 113 can use a generative artificial intelligence model to generate a list of reasons explaining the extrinsic load score along with the extrinsic load score. Further detail about the calculation of the extrinsic load score is included in the description of FIG. 3.

At block 209, the cognitive load engine 113 can be executed to calculate an intrinsic load score. The intrinsic load score can be a measure of the intrinsic cognitive load experienced by the user during the user interactions 119 from block 200. In some examples, the cognitive load engine 113 can calculate the intrinsic load score based at least in part on the user interactions 119 from block 200. In some examples, the cognitive load engine 113 can calculate the intrinsic load score based at least in part on other data. The cognitive load engine 113 can use a generative artificial intelligence model to generate a list of reasons explaining the intrinsic load score along with the intrinsic load score. Further detail about the calculation of the intrinsic load score is included in the description of FIG. 4.

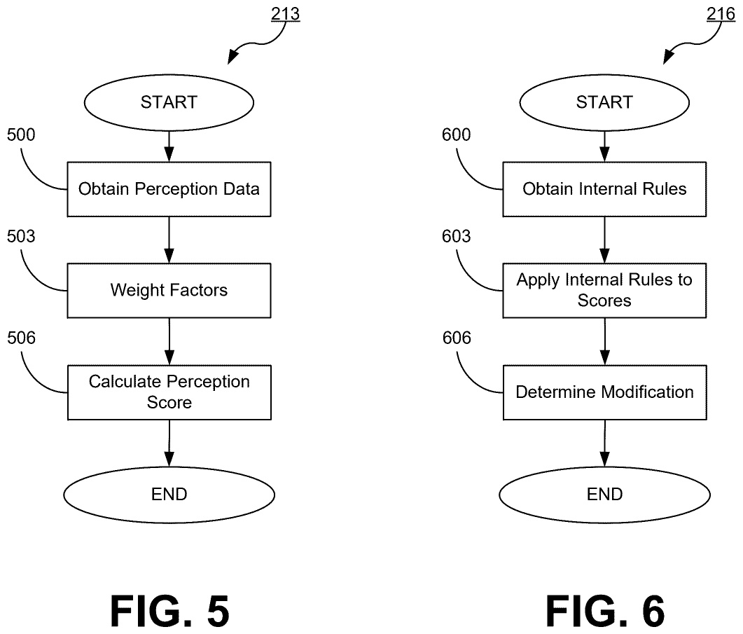

Next, at block 213, the cognitive load engine 113 can be executed to calculate a perception score. The perception score can be representative of a measurement of the cognitive load on a user due to their perception of the organization. In some examples, the cognitive load engine 113 can calculate the perception score based at least in part on the user interactions 119 from block 200 or based at least in part on other data. The cognitive load engine 113 can use a generative artificial intelligence model to generate a list of reasons explaining the perception load score along with the perception load score. Further detail about the calculation of the perception load score is included in the description of FIG. 5.

At block 216, the cognitive load engine 113 can be executed to conduct an analysis of one or more of the extrinsic load score, the intrinsic load score, and the perception score. In some examples, the analysis can be based at least in part on the scores and based at least in part on the list of reasons explaining the respective scores. The cognitive load engine 113 can conduct the analysis based at least in part on the intended task identified at block 203. Further detail about the analysis is included in the description of FIG. 6.

At block 219, the cognitive load engine 113 can be executed to modify a user experience. The cognitive load engine 113 can modify a user experience based at least in part on the analysis conducted at block 216. For example, the cognitive load engine 113 can determine from the analysis that the user needs to be directed to a service representative instead of a chat bot and cause the user to be redirected to the service representative. In another example, the cognitive load engine 113 can determine that a particular service or product would be suited for the user and cause the service or product to be promoted to the user. The cognitive load engine 113 can modify the user experience by modifying the steps necessary to complete the intended task from block 203, redirecting the user to another platform, adding or removing pop-ups and advertisements, or otherwise altering the journey for the user. After block 219, the flowchart of FIG. 2 can end.

Moving now to FIG. 3, shown is a flowchart that provides one example of the operation of the cognitive load engine 113 at block 206 from FIG. 2. The flowchart of FIG. 3 provides merely an example of the many different types of functional arrangements that can be employed to implement the operation of block 206 of the cognitive load engine 113 of FIG. 2. As an alternative, the flowchart of FIG. 3 can be viewed as depicting an example of elements of a method implemented within the network environment 100.

Beginning with block 300, the cognitive load engine 113 can be executed to determine relevancy. The cognitive load engine 113 can determine the content that is being presented to the user during the journey and based at least in part on the user interactions 119 from block 200 of FIG. 2, the cognitive load engine 113 can determine a relevancy of the content. In some examples, the cognitive load engine 113 can determine the relevancy of the content based at least in part on the intended task identified at block 203 of FIG. 2. For example, if the cognitive load engine 113 determined that a user is intending to extend a credit limit for an account, the cognitive load engine 113 can evaluate the relevancy of the search results based at least in part on how many of the results directly correspond to extending a credit limit.

Next, at block 303, the cognitive load engine 113 can be executed to weight one or more factors. The cognitive load engine 113 can use a weighted equation in order to calculate the extrinsic load score. Before calculating the score, the cognitive load engine 113 can first determine the appropriate weights to apply to each factor of the equation. In some examples, the cognitive load engine 113 can weight each factor of the equation based at least in part on the relevancy determined at block 300. In some examples, the cognitive load engine 113 weights the factors based at least in part on the user interactions from block 200 of FIG. 2. For example, the cognitive load engine 113 can apply a higher weight to a user's fixation on the content than for the relevancy of the content since the user's attention is more demonstrative of the extrinsic load than the predicted relevancy of the content.

At block 306, the cognitive load engine 113 can be executed to calculate an extrinsic load score. The cognitive load engine 113 can use the weighted factors from block 303 in the equation to calculate an extrinsic load score. In some examples, the equation is a linear equation, with each factor having a particular weight determined above. In some examples, the cognitive load engine 113 using a machine learning model to calculate the extrinsic load score based at least in part on the relevancy of the content determined at block 300. The cognitive load engine 113 can use the user interactions 119 and/or the intended task in order to calculate the extrinsic load score. After block 306, the flowchart of FIG. 3 can end.

Moving now to FIG. 4, shown is a flowchart that provides one example of the operation of the cognitive load engine 113 at block 209 from FIG. 2. The flowchart of FIG. 4 provides merely an example of the many different types of functional arrangements that can be employed to implement the operation of block 209 of the cognitive load engine 113 of FIG. 2. As an alternative, the flowchart of FIG. 4 can be viewed as depicting an example of elements of a method implemented within the network environment 100.

Beginning with block 400, the cognitive load engine 113 can be executed to obtain user data 126. In some examples, the cognitive load engine 113 can access a data store 116 to obtain user data 126. The cognitive load engine 113 can also obtain the user data 126 from the client device 106 or another system, service, or application in the network environment 100. In some examples, the cognitive load engine 113 can obtain the user data 126 based at least in part on the intended task identified at block 203 of FIG. 2. For example, if the intended task is identified as extending a credit limit and the user has engaged with a search engine to accomplish this task, the cognitive load engine 113 can obtain user data 126 related to the user's past interactions with the search engine.

At block 403, the cognitive load engine 113 can be executed to weight one or more factors. The cognitive load engine 113 can use a weighted equation in order to calculate the intrinsic load score. Before calculating the score, the cognitive load engine 113 can first determine the appropriate weights to apply to each factor of the equation. In some examples, the cognitive load engine 113 can weight each factor of the equation based at least in part on the user data 126 obtained at block 400. In some examples, the cognitive load engine 113 weights the factors based at least in part on demographic data 123 as well. For example, the cognitive load engine 113 can apply a higher weight to the number of the user's past interactions with the search engine than to the user's birth generation since actual experience with the application may be more demonstrative of the user's savviness with the technology than the presumed experience based on generation.

Next, at block 406, the cognitive load engine 113 can be executed to calculate an intrinsic load. The cognitive load engine 113 can use the weighted factors from block 403 in the equation to calculate an intrinsic load score. In some examples, the equation is a linear equation, with each factor having a particular weight determined above. In some examples, the cognitive load engine 113 using a machine learning model to calculate the intrinsic load score based at least in part on the user data 126 determined at block 400. The cognitive load engine 113 can use the user interactions 119 as well as demographic data 123 in order to calculate the intrinsic load score. After block 406, the flowchart of FIG. 4 can end.

Moving now to FIG. 5, shown is a flowchart that provides one example of the operation of the cognitive load engine 113 at block 213 from FIG. 2. The flowchart of FIG. 5 provides merely an example of the many different types of functional arrangements that can be employed to implement the operation of block 213 of the cognitive load engine 113 of FIG. 2. As an alternative, the flowchart of FIG. 5 can be viewed as depicting an example of elements of a method implemented within the network environment 100.

Beginning with block 500, the cognitive load engine 113 can be executed to obtain perception data 139. The perception data 139 can be obtained from a data store 116, the client device 106, or another system, service, or application in the network environment 109. In some examples, the cognitive load engine 113 can obtain the perception data 139 by means of one or more prompts sent to a client application 146 and responses returned from the client application 146. For example, the cognitive load engine 113 can obtain perception data 139 by means of a survey. In another example, the cognitive load engine 113 can obtain perception data 139 from user data 136 regarding the user's past interactions with particular brands.

Next, at block 503, the cognitive load engine 113 can be executed to weight factors. The cognitive load engine 113 can use a weighted equation in order to calculate the perception score. Before calculating the score, the cognitive load engine 113 can first determine the appropriate weights to apply to each factor of the equation. In some examples, the cognitive load engine 113 can weight each factor of the equation based at least in part on the perception data 139 obtained at block 500. In some examples, the cognitive load engine 113 weights the factors based at least in part on demographic data 123 as well. For example, the cognitive load engine 113 can weight a user's social media commentary higher than the number of brand-specific adds the user has seen because the commentary may be more indicative of a user's perception than what they are exposed to online.

At block 506, the cognitive load engine 113 can be executed to calculate a perception score. The cognitive load engine 113 can use the weighted factors from block 503 in the equation to calculate a perception score. In some examples, the equation is a linear equation, with each factor having a particular weight determined above. In some examples, the cognitive load engine 113 using a machine learning model to calculate the perception score based at least in part on the perception data 139 obtained at block 500. The cognitive load engine 113 can use the user interactions 119 as well as demographic data 123 in order to calculate the perception score. After block 506, the flowchart of FIG. 5 can end.

Moving now to FIG. 6, shown is a flowchart that provides one example of the operation of the cognitive load engine 113 at block 216 from FIG. 2. The flowchart of FIG. 6 provides merely an example of the many different types of functional arrangements that can be employed to implement the operation of block 216 of the cognitive load engine 113 of FIG. 2. As an alternative, the flowchart of FIG. 6 can be viewed as depicting an example of elements of a method implemented within the network environment 100.

Beginning with block 600, the cognitive load engine 113 can be executed to obtain internal rules 136. The internal rules 136 can be obtained from a data store 116, the client device 106, or another system, service, or application in the network environment 109. In some examples, the cognitive load engine 113 can obtain the internal rules 136 in response to calculating one or more of the extrinsic load score, the intrinsic load score, and the perception score.

Next, at block 603, the cognitive load engine 113 can be executed to apply the internal rules 136 to the scores. In some examples, the cognitive load engine 113 can apply the internal rules 136 to one or more of the extrinsic load score, the intrinsic load score, and the perception score. Applying the internal rules 136 can include evaluating the scores in view of the internal rules 136 and comparing the scores to threshold values within the internal rules 136. In some examples, the cognitive load engine 113 can use a machine learning model to apply the internal rules 136 to the scores in order to make determinations about the scores. For example, the cognitive load engine 113 can determine that the extrinsic load score exceeds a threshold value in the internal rules 136 which requires intervention to reduce the score. Similarly, the cognitive load engine 113 can determine that the reasons for an intrinsic load score is below a threshold value in the internal rules 136 and that no intervention is needed.

At block 606, the cognitive load engine 113 can be executed to determine a modification. The cognitive load engine 113 can determine a modification which should be made to the user experience based at least in part on the internal rules 136 obtained at block 600. In some examples, the cognitive load engine 113 can determine the modification based at least in part on the application of the internal rules 136 to the scores at block 603. The cognitive load engine 113 can use a machine learning model to determine a modification that should be made to the user experience. Determining the modification can include steps such as identifying an appropriate modification from a list of potential modifications or using generative artificial intelligence to suggest a new modification. For example, the cognitive load engine 113 can determine that since the extrinsic load score exceeds a threshold value of the internal rules 136 and the reasons for the high extrinsic load score are related to relevancy of the content, redirecting the user to a chat with an agent or chat bot will reduce the extrinsic load on the user and expedite the user experience. After block 606, the flowchart of FIG. 6 can end.

A number of software components previously discussed are stored in the memory of the respective computing devices and are executable by the processor of the respective computing devices. In this respect, the term “executable” means a program file that is in a form that can ultimately be run by the processor. Examples of executable programs can be a compiled program that can be translated into machine code in a format that can be loaded into a random access portion of the memory and run by the processor, source code that can be expressed in proper format such as object code that is capable of being loaded into a random access portion of the memory and executed by the processor, or source code that can be interpreted by another executable program to generate instructions in a random access portion of the memory to be executed by the processor. An executable program can be stored in any portion or component of the memory, including random access memory (RAM), read-only memory (ROM), hard drive, solid-state drive, Universal Serial Bus (USB) flash drive, memory card, optical disc such as compact disc (CD) or digital versatile disc (DVD), floppy disk, magnetic tape, or other memory components.

The memory includes both volatile and nonvolatile memory and data storage components. Volatile components are those that do not retain data values upon loss of power. Nonvolatile components are those that retain data upon a loss of power. Thus, the memory can include random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, or other memory components, or a combination of any two or more of these memory components. In addition, the RAM can include static random access memory (SRAM), dynamic random access memory (DRAM), or magnetic random access memory (MRAM) and other such devices. The ROM can include a programmable read-only memory (PROM), an erasable programmable read-only memory (EPROM), an electrically erasable programmable read-only memory (EEPROM), or other like memory device.

Although the applications and systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowcharts show the functionality and operation of an implementation of portions of the various embodiments of the present disclosure. If embodied in software, each block can represent a module, segment, or portion of code that includes program instructions to implement the specified logical function(s). The program instructions can be embodied in the form of source code that includes human-readable statements written in a programming language or machine code that includes numerical instructions recognizable by a suitable execution system such as a processor in a computer system. The machine code can be converted from the source code through various processes. For example, the machine code can be generated from the source code with a compiler prior to execution of the corresponding application. As another example, the machine code can be generated from the source code concurrently with execution with an interpreter. Other approaches can also be used. If embodied in hardware, each block can represent a circuit or a number of interconnected circuits to implement the specified logical function or functions.

Although the flowcharts show a specific order of execution, it is understood that the order of execution can differ from that which is depicted. For example, the order of execution of two or more blocks can be scrambled relative to the order shown. Also, two or more blocks shown in succession can be executed concurrently or with partial concurrence. Further, in some embodiments, one or more of the blocks shown in the flowcharts can be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein that includes software or code can be embodied in any non-transitory computer-readable medium for use by or in connection with an instruction execution system such as a processor in a computer system or other system. In this sense, the logic can include statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. Moreover, a collection of distributed computer-readable media located across a plurality of computing devices (e.g., storage area networks or distributed or clustered filesystems or databases) may also be collectively considered as a single non-transitory computer-readable medium.

The computer-readable medium can include any one of many physical media such as magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium can be a random access memory (RAM) including static random access memory (SRAM) and dynamic random access memory (DRAM), or magnetic random access memory (MRAM). In addition, the computer-readable medium can be a read-only memory (ROM), a programmable read-only memory (PROM), an erasable programmable read-only memory (EPROM), an electrically erasable programmable read-only memory (EEPROM), or other type of memory device.

Further, any logic or application described herein can be implemented and structured in a variety of ways. For example, one or more applications described can be implemented as modules or components of a single application. Further, one or more applications described herein can be executed in shared or separate computing devices or a combination thereof. For example, a plurality of the applications described herein can execute in the same computing device, or in multiple computing devices in the same computing environment 103.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

Therefore, the following is claimed:

1. A system, comprising:
a computing device comprising a processor and a memory; and
machine-readable instructions stored in the memory that, when executed by the processor, cause the computing device to at least:
monitor a plurality of user interactions associated with a user;
identify an intended task associated with one or more user interactions of the plurality of user interactions;
calculate an extrinsic load score based at least in part on the plurality of user interactions;
conduct an analysis of the extrinsic load score based at least in part on the intended task; and
modify a user experience based at least in part on the analysis.

2. The system of claim 1, wherein the machine-readable instructions which cause the computing device to calculate the extrinsic load score, further cause the computing device to at least calculate the extrinsic load score based at least in part on the intended task.

3. The system of claim 1, wherein the machine-readable instructions further cause the computing device to at least:
obtain user data associated with the user; and
calculate an intrinsic load score based at least in part on the user data.

4. The system of claim 3, wherein the analysis is further based at least in part on the intrinsic load score.

5. The system of claim 1, wherein the machine-readable instructions which cause the computing device to conduct the analysis of the extrinsic load score further cause the computing device to at least apply a set of internal rules to the plurality of user interactions and the extrinsic load score.

6. The system of claim 1, wherein the machine-readable instructions which cause the computing device to modify the user experience further cause the computing device to at least cause a user to be directed to a service representative.

7. The system of claim 1, wherein the machine-readable instructions further cause the computing device to at least:
obtain perception data associated with the user; and
calculate a perception score based at least in part on the perception data.

8. A method, comprising:
monitoring, by a computing device, a plurality of user interactions associated with a user;
identifying, by the computing device, an intended task associated with one or more user interactions of the plurality of user interactions;
calculating, by the computing device, an extrinsic load score based at least in part on the plurality of user interactions;
conducting, by the computing device, an analysis of the extrinsic load score based at least in part on the intended task; and
modifying, by the computing device, a user experience based at least in part on the analysis.

9. The method of claim 8, wherein calculating the extrinsic load score is further based at least in part on the intended task.

10. The method of claim 8, further comprising:
obtaining, by the computing device, user data associated with the user; and
calculating, by the computing device, an intrinsic load score based at least in part on the user data.

11. The method of claim 10, wherein the analysis is further based at least in part on the intrinsic load score.

12. The method of claim 8, wherein conducting the analysis of the extrinsic load score further comprises at least applying, by the computing device, a set of internal rules to the plurality of user interactions and the extrinsic load score.

13. The method of claim 8, wherein modifying the user experience further comprises at least causing, by the computing device, a user to be directed to a service representative.

14. The method of claim 8, further comprising:
obtaining, by the computing device, perception data associated with the user; and
calculating, by the computing device, a perception score based at least in part on the perception data.

15. A system, comprising:
a computing device comprising a processor and a memory; and
machine-readable instructions stored in the memory that, when executed by the processor, cause the computing device to at least:
monitor a plurality of user interactions associated with a user;
calculate an extrinsic load based at least in part on the plurality of user interactions;
calculate an intrinsic load based at least in part on user data associated with the user;
conduct an analysis of the extrinsic load and the intrinsic load; and
modify a user experience based at least in part on the analysis of the extrinsic load and the intrinsic load.

16. The system of claim 15, wherein the machine-readable instructions which cause the computing device to calculate an extrinsic load further cause the computing device to at least:
determine a relevancy of the plurality of user interactions;
weight a plurality of factors based at least in part on the relevancy; and
calculate the extrinsic load based at least in part on a plurality of weighted factors.

17. The system of claim 15, wherein the machine-readable instructions which cause the computing device to calculate an intrinsic load further cause the computing device to at least:
obtain user data associated with the user;
weight a plurality of factors based at least in part on the user data and the plurality of user interactions; and
calculate the intrinsic load based at least in part on a plurality of weighted factors.

18. The system of claim 15, wherein the machine-readable instructions further cause the computing device to at least:
obtain perception data;
weight a plurality of factors based at least in part on the perception data; and
calculate a perception score based at least in part on a plurality of weighted factors.

19. The system of claim 15, wherein the machine-readable instructions which cause the computing device to conduct the analysis of the extrinsic load and the intrinsic load, further cause the computing device to at least:
obtain a set of internal rules;
apply the set of internal rules to the extrinsic load and the intrinsic load; and
determine a modification to apply to the user experience.

20. The system of claim 15, wherein the machine-readable instructions further cause the computing device to at least:
identify an intended task associated with one or more user interactions of the plurality of user interactions; and
calculate the extrinsic load based at least in part on the intended task.
